(19) United States
(12) Patent Application Publication
(10) Pub. No.: US 2014/0271815 A1
(43) Pub. Date: Sep. 18, 2014

(54) HEAT-AND FREEZE-STABLE VACCINES AND METHODS OF MAKING AND USING SAME

(71) Applicants: Aryo Sorayya, Danville, CA (US); Rajiv Nayar, Danville, CA (US); Mitra Mosharraf, Danville, CA (US)

(72) Inventors: Aryo Sorayya, Danville, CA (US); Rajiv Nayar, Danville, CA (US); Mitra Mosharraf, Danville, CA (US)

(21) Appl. No.: 13/837,637

(22) Filed: Mar. 15, 2013

(51) Int. Cl. A61K 47/24; A61K 9/51; A61K 9/127; A61K 47/08; A61K 39/00

(52) U.S. Cl. CPC A61K 47/24; A61K 47/08; A61K 39/0005; A61K 9/127; A61K 9/5123
USPC 424/450; 424/184.1; 424/489

(57) ABSTRACT

A vaccine adjuvant composition comprising: a lipid selected from the group consisting of: Dipalmitoyl phosphatidlcholine (DPPC), Dipalmitoyl phosphatidylglycerol (DPPG), Dioleoyl phosphatidylcholine (DOPC), and cholesterol and containing a positively or negatively charged lipid with associated/entrapped protein antigen.

### CROSS REFERENCE TO RELATED APPLICATIONS

This application is a continuation of Provisional U.S. patent application No. 61/611, 997 filed Mar. 16, 2012.

### BACKGROUND OF THE INVENTION

The invention relates to injectable liposomal vaccines that is comprised of natural lipids, dipalmitoyl phosphatidlcholine (DPPC), dioleoyl phosphatidylcholine (DOPC), and cholesterol with either a negative charged lipid (e.g. dipalmitoyl phosphatidylglycerol (DPPG) or with a positively charged lipid:octadecylamine (Stearylamine, SA)).

The cold-chain, a supply chain for pharmaceutical drugs based on temperature control, is a laborious process that attempts to keep vaccines at the suggested 2-8° C. range, and thus costs companies and organizations (e.g. UNICEF) millions of dollars every year. Freeze-sensitive vaccines represent over 30% of the $439 million UNICEF spent on all vaccines in 2005 and the $757 million spent in 2010. Carrying-containers using ice (prominent in third world countries), defective refrigerators, and extreme cold climates can impel these vaccines to freeze and render them ineffective. Rate of exposure to freezing temperatures in developed and developing countries is 13.5% and 21.9%, respectively—making this a global concern. Freezing is a risk at any level of the cold chain, and serves as a major problem for many salient vaccines, including Hepatitis A/B, Diphtheria and Tetanus Toxoids, and Haemophilus influenzae type B.

Currently, aluminum-based adjuvants (e.g. Aluminum-Phosphate, Aluminum hydroxide) dominate their field and, prior to 2009, were the only licensed adjuvants in the U.S.; however, these inorganic adjuvants face numerous problems as they are frost sensitive and not readily lyophilizable. The limitations placed on vaccines by adjuvants that are not freeze-compatible severely restrict the use of such vaccines and make them unavailable in many areas in the world.

Furthermore, these freeze sensitive adjuvants have further failed to elicit adequate immune responses in many cases and also do not bind effectively to all protein antigens. This has spurred interest in other forms of adjuvants—mainly liposomes—which are much more versatile. Indeed, in the 1,316 publications about liposomal vaccines since 1974, one quarter have been published in the past three years, and have propelled the creation of multiple vaccines using liposomal adjuvants in area such as Influenza and Hepatitis A. Inflexal V and Epaxal, respectively, are two liposome-based vaccines approved for human use. Both of these vaccines must be stored at 2-8° C. and should not be frozen. Other liposomal vaccines that are currently moving toward regulatory approval are mainly based on synthetic, cationic lipids which are in general not sufficiently immunogenic, and are thus often combined with immunostimulators such as lipid A.

### SUMMARY OF THE INVENTION

In one aspect, a composition includes Dipalmitoyl phosphatidlcholine (DPPC), Dioleoyl phosphatidylcholine (DOPC), cholesterol (Chol); and a charged lipid with an entrapped or adsorbed protein antigen induce antibody response against an antigen.

Implementations of the above aspect can include one or more of the following. Liposomes or nano-particles consisting of the novel composition comprising of Dipalmitoyl phosphatidlcholine (DPPC), Dioleoyl phosphatidylcholine (DOPC), and cholesterol (Chol) and a charged lipid, with an entrapped or adsorbed protein antigen induce antibody response against the antigen. Liposomes or nano particles in claim 1 can be used as a new vaccine adjuvant. Liposomes or nano particles in claim 1 do not lose their immunogenicity after being exposed to freezing temperature during multiple freeze-thaws and are freeze-stable adjuvants. The composition is preferably in a molar ratio of 40:25:20 of DPPC:DOPC:Chol and 15 for a negatively charged lipid Dipalmitoyl phosphatidylglycerol (DPPG). The composition is preferably in a molar ratio of 40:25:20 of DPPC:DOPC:Chol and 15 for a positively charged lipid Stearylamine (SA). The mean hydrodynamic particle diameter of particles in claim 1 is in the size range of 300-1000 nm. The solution containing the particles remains immunogenic after lyophilization during which it is frozen to −40° C. to −50° C. The adjuvant particles can be used as an alternative to freeze-sensitive aluminum salt adjuvants in all vaccine formulations containing these adjuvants. The protein antigen can be selected from or derived from the group consisting of rotavirus, foot and mouth disease virus, influenza A virus, influenza B virus, influenza C virus, H1N1, H2N2, H3N2, H5N1, H7N7, H1N2, H9N2, H7N2, H7N3, H10N7, human parainfluenza type 2, herpes simplex virus, Epstein-Barr virus, varicella virus, porcine herpesvirus 1, cytomegalovirus, lyssavirus, Bacillus anthracis, anthrax PA and derivatives, poliovirus, Hepatitis A, Hepatitis B, Hepatitis C, Hepatitis E, distemper virus, Venezuelan equine encephalomyelitis, feline leukemia virus, reovirus, respiratory syncytial virus, Lassa fever virus, polyoma tumor virus, canine parvovirus, papilloma virus, tick borne encephalitis virus, rinderpest virus, human rhinovirus species, Enterovirus species, Mengovirus, paramyxovirus, avian infectious bronchitis virus, human T-cell leukemia-lymphoma virus 1, human immunodeficiency virus-1, human immunodeficiency virus-2, lymphocytic choriomeningitis virus, parvovirus B19, adenovirus, rubella virus, yellow fever virus, dengue virus, bovine respiratory syncitial virus, corona virus, Bordetella pertussis, Bordetella bronchiseptica, Bordetella parapertussis, Brucella abortis, Brucella melitensis, Brucella suis, Brucella ovis, Brucella species, Escherichia coli, Salmonella species, Salmonella typhi, Streptococci, Vibrio cholera, Vibrio parahaemolyticus, Shigella, Pseudomonas, tuberculosis, avium, Bacille Calmette Guerin, Mycobacterium leprae, Pneumococci, Staphylococci, Enterobacter species, Rochalimaia henselae, Pasteurella haemolytica, Pasteurella multocida, Chlamydia trachomatis, Chlamydia psittaci, Lymphogranuloma venereum, Treponema pallidum, Haemophilus species, Mycoplasma bovigenitalium, Mycoplasma pulmonis, Mycoplasma species, Borrelia burgdorferi, Legionalla pneumophila, Colstridium botulinum, Corynebacterium diphtheriae, Yersinia entercolitica, Rickettsia rickettsii, Rickettsia typhi, Rickettsia prowsaekii, Ehrlichia chaffeensis, Anaplasma phagocytophilum, Plasmodium falciparum, Plasmodium vivax, Plasmodium malariae, Schistosomes, trypanosomes, Leishmania species, Filarial nematodes, trichomoniasis, sarcosporidiasis, Taenia saginata, Taenia solium, Leishmania, Toxoplasma gondii, Trichinella spiralis, coccidiosis, Eimeria tenella, Cryptococcus neoformans, Candida albican, Apergillus fumigatus, coccidioidomycosis, Neisseria gonorrhoeae, malaria circumsporozoite protein, malaria merozoite protein, trypanosome surface antigen protein, pertussis, alphaviruses, adenovirus, diphtheria toxoid, tetanus toxoid, meningococcal outer membrane protein, streptococcal M protein, Influenza hemagglutinin, cancer antigen, tumor antigens, toxins, Clostridium perfringens epsilon toxin, ricin toxin, pseudomonas exotoxin, exotoxins, neurotoxins, cytokines, cytokine receptors, monokines, monokine receptors, plant pollens, animal dander, and dust mites. The particles can be used in formulation of vaccines against diphtheria, tetanus, pertussis (whooping cough), influenza, hepatitis B, botulinium toxin, anthrax, or combination vaccines such as, PedvaxHlB (Haemophilus b Conjugate and Meninococcal Protein Conjugate), COMVAX® (Meningococcal Protein Conjugate and Hepatitis B, recombinant antigens), TRIPEDIA® (Diphtheria and Tetanus Toxoids and Acellular Pertussis antigens). INFANRIX (Diphtheria and Tetanus Toxoids and Acellular Pertussis antigens), or any other vaccines that loses its potency upon freezing.

Advantages of the above aspect may include one or more of the following. Using a new lipid composite as an adjuvant, users can manufacture vaccines with entrapped protein antigen that had significant immunogenic response in mice. This lipid composite did not lose its immunogenic activity upon freezing and lyophilization and might thus be used as a freeze-stable vaccine as an alternative to Aluminum salt adjuvants. The liposomal vaccines described here do not require any additional co-adjuvant such as Lipid A, Lipid A derivatives, monophosphoryl lipid A, monophosphoryl lipid A derivatives, lipopolysaccharide, muramyl dipeptide, CpG containing oligonucleotides, TLR-4 agonists, flagellin, flagellins derived from gram negative bacteria, TLR-5 agonists, fragments of flagellins capable of binding to TLR-5 receptors, saponins, analogues of saponins, QS-21, purified saponin fractions, ISCOMS and saponin combinations with sterols to render the liposomes immunogenic. The liposomes with the associated antigen retain their immunogenicity after exposure to freezing temperatures, after multiple freeze-thaws, and after being freeze-dried or lyophilized. The liposomal adjuvants did not lose their immunogenic activity after multiple freeze-thaw cycles and also additional freezing to −40° C. during lyophilization and might thus be used in vaccine formulations as an alternative to aluminum salt adjuvants. The vaccine adjuvants designed provide a technological platform for development of immunogenic, freeze-stable vaccines, preventing product damage during accidental freezing in the cold chain. These liposomal vaccine adjuvants can be used to develop vaccines that are stable against freezing. The novel freeze-dried liposomal vaccine described in Example 1 demonstrated efficacy similar to that of a liquid aluminum-phosphate based vaccine as measured by the antibody response to chicken egg Lysozyme in mice.

Such liposomal vaccine products offer numerous advantages over Aluminum-based vaccines in regards to safety, freeze-stability, tolerability, biodegradability, and versatility. Hence, this unique liposomal based adjuvant can be employed instead of Aluminum adjuvants in current freeze sensitive vaccines against for example diphtheria, tetanus, pertussis (whooping cough), influenza and anthrax. The novel vaccine adjuvant designed thus provides a technological platform for development of immunogenic, freeze-stable vaccines, preventing product damage during accidental freezing in the cold chain.

### BRIEF DESCRIPTION OF THE DRAWINGS

FIG. 1 shows an exemplary immune response (450 nm absorbance) of the various formulations at different dilutions of the sera—80,000, 160,000, and 320.000-fold. The amount of absorbance at 450 nm reflects the amount of antibody present in the sera.

FIG. 2 shows an exemplary Standard Curve of Mouse Anti-Lysozyme Antibody.

FIG. 3 shows an exemplary Average Amount of Mouse Anti-Lysozyme Antibody for Different Formulations (mg/mL)

FIGS. 4-5 show exemplary Average Amount of Mouse Anti-Tetanus Toxoid Antibody for Different Formulations (U/mL), where the average titers of mouse anti-Tetanus Toxid IgG from 5 mice are compared among the formulations (FIG. 5).

### DESCRIPTION

The immunogenicity of a new liposomal adjuvant consisting of a lipid blend composition comprising the following lipids: Dipalmitoyl phosphatidylcholine (DPPC), Dioleoyl phosphatidylcholine (DOPC), Cholesterol was tested with either a negative charge lipid: Dipalmitoyl phosphatidylglycerol (DPPG) in Example 1 or with a positively charged lipid: Octadecylamine (Stearylamine, SA) in Example 2. In these embodiments the molar ratio of DPPC:DOPC:cholesterol was 40:25:20 and that of the charged lipid was 15. Chicken egg lysozyme and tetanus light chain (TLC) were used as model protein antigens, respectively. The charged liposomal adjuvants with the associated protein antigen were immunogenic and did not lose their immunogenic activity after multiple freeze-thaw cycles and also additional freezing to −40° C. during lyophilization. Thus, they can be used in vaccine formulations as an alternative to Aluminum salt adjuvants.

The vaccine adjuvant designed provides a technological platform for development of immunogenic, freeze-stable vaccines, preventing product damage during accidental freezing in the cold chain. The liposomal vaccine adjuvant can be used to develop vaccines that are stable against freezing. The novel freeze-dried liposomal vaccine demonstrated efficacy similar to that of a liquid aluminum-phosphate based vaccine as measured by the antibody response to chicken egg Lysozyme in mice. The results showed that the liposomal vaccine was freeze-stable and did not lose its immunogenic activity upon freezing/freeze-drying. Such a liposomal vaccine product offers numerous advantages over Aluminum-based vaccines in regards to safety, freeze-stability, tolerability, biodegradability, and versatility. It is recommended to use this adjuvant instead of Aluminum adjuvants in current freeze sensitive vaccines against for example diphtheria, tetanus, pertussis (whooping cough), influenza and anthrax. The novel vaccine adjuvant designed thus provides a technological platform for development of immunogenic, freeze-stable vaccines, preventing product damage during accidental freezing in the cold chain.

### Example 1

### Vaccine Preparation

Four different formulations are summarized as it is shown in Table 1:

TABLE 1
Formulation descriptions

| Formulation | Adjuvant | Antigen | Stabilizer | Dosage form |
|---|---|---|---|---|
| 1 | DPPC/DPPG/ | Chicken Egg | Sucrose | Liquid |
| | DOPC/ | Lysozyme | | |
| | Cholesterol | | | |
| 2 | DPPC/DPPG/ | Chicken Egg | Sucrose | Lyophilized |
| | DOPC/ | Lysozyme | | |
| | Cholesterol | | | |
| 3 | Aluminum | Chicken Egg | Sucrose | Liquid |
| | phosphate | Lysozyme | | |
| | (Adju-Phos) | | | |
| 4 | None | Chicken Egg | Sucrose | Liquid |
| | | Lysozyme | | |

### Preparation of Liquid and Lyophilized Liposomal Vaccines (Formulations 1 & 2)

Specific amounts of DPPC, DOPC, cholesterol and DPPG in a molar ratio of 40:25:20:15 were dissolved in a co-solvent containing chloroform, methanol, and water. The lipid blend was dried in a 45° C. water bath under a stream of nitrogen gas, and any remaining residual solvent was removed under vacuum overnight to ensure complete desiccation.

The dried lipid-blend was hydrated in 1 mL of a 9 mg/mL Lysozyme stock solution and vortexed to form Multi Lamellar Vesicles (MLVs). In order to improve the entrapping efficacy, the liposome solution was freeze-thawed five times using an acetone-ice bath and a warm-water bath. The liposome mixture was centrifuged at 14,000 rpm for 20 min to separate the liposomes from the unbound Lysozyme solution. The unbound lyozyme in supernatant was removed and the amount of liposome associated protein determined.

The liposome pellet containing entrapped Lysozyme was resuspended in 1 mL of a sterile filtered 10% w/w sucrose solution and vortexed. To reduce the size of liposomes and to obtain a more homogenous particle distribution, the liposome solution was extruded five times through two 800 nm polycarbonate filters in a 10 mL extruder using nitrogen gas at pressures around 100 psi. Lysozyme concentration in the concentrated liposome solution was determined and adjusted to 200 ug/mL by diluting the solution in 10% sucrose.

Eight vials were filled with 1 mL of liposome solution each. Four of the vials were freeze-dried in a lyophilizer by first freezing the solution at −45° C. and then subliming the ice at −20° C. under high vacuum. Secondary drying was performed at 25° C. shelf temperature to remove any residual water from the cake.

Preparation of 200 ug/mL Lysozyme/Adju-Phos Vaccines (Formulation 3)

1.0 mL of Lysozyme stock solution was added to 1 mL Adju-Phos. The solution was mixed and stored for 40 minutes at room temperature to allow for Lysozyme to be adsorbed by Adju-Phos. The solution was centrifuged for 5 min at 5,000 RPM to precipitate the complex. The supernatant containing unbound lysozyme was removed. The concentration of unbound and bound Lysozyme was determined using UV Spectrophotometry. Based on the amount of bound Lysozyme, an adequate amount of a 10% sucrose solution was added to the Adju-Phos/Lysozyme pellet to acquire a 200 ug/mL final concentration of Lysozyme

Preparation of 200 ug/mL Lysozyme in 10% Sucrose (Formulation 4)

In order to prepare a Lysozyme solution with no adjuvant, 10 mL 200 ug/mL Lysozyme solution was prepared by diluting a specific amount of the Lysozyme stock solution with 10% sucrose.

### Particle Size Characterization Using Dynamic Light Scattering (DLS)

The particle sizes of the liposome preparations were determined by dynamic light scattering analysis. Each sample was analyzed in triplicate. The mean hydrodynamic diameter by intensity and standard deviation (SD) were calculated (n=3) using the DLS software.

### Mice Immunization

100 ul (2×50 ul) of each formulation was injected intramuscularly (im) in the shoulder of four female CD-1 mice (Charles River, Hollister Calif.). A booster shot was administered on day 14. Totally, twenty mice were tested in groups of four. 16 were used for vaccine testing and 4 were naïve mice as negative control. All of the animals were observed immediately after dosing and daily thereafter. On Day 28, serum was collected from the immunized mice as well as the control group and the antibody response to each vaccine was determined by Indirect Enzyme-Linked Immunosorbent Assay (Indirect ELISA) to chicken egg lysozyme.

Statistical analysis of the induced immune responses was performed, including a two-tail t-test. Differences were considered significant if p<0.05.

The study design and group designation are summarized in Table 2.

TABLE 2
Study Group Designations

| | | | | Study Event | | |
|---|---|---|---|---|---|---|
| Group/ | | | | | | Day 28 - |
| Test | No. mice/ | Dose | | | Day 14 - | Terminal |
| Article # | group | Volume | Sex | Day 0 - Dose | Dose | Bleed |
| 1 | 4 | 100 μL in | F | Intramuscular | Intramuscular | Cardiac |
| | | two sites | | | | Puncture |
| 2 | 4 | 100 μL in | F | Intramuscular | Intramuscular | Cardiac |
| | | two sites | | | | Puncture |
| 3 | 4 | 100 μL in | F | Intramuscular | Intramuscular | Cardiac |
| | | two sites | | | | Puncture |
| 4 | 4 | 100 μL in | F | Intramuscular | Intramuscular | Cardiac |
| | | two sites | | | | Puncture |

| | | | | | |
|---|---|---|---|---|---|
| 5 (naïve | 4 | N/A | F | None - Naïve Animals | Cardiac |
| animals) | | | | | Puncture |

Prior to dosing, animals will be arbitrarily assigned to treatment groups. On Day 0, each mouse will be injected intramuscularly with 100 μL of test article (50 μL in each shoulder) using an appropriate size syringe and beveled needle (i.e. 1 cc insulin syringe w/26 gauge (or smaller) needle).
Group 1 was dosed with Formulation 1 (Liposomes+Lysozyme); Group 2 was dosed with Formulation 2 (Lyophilized Liposomes+Lysozyme); Group 3 was dosed with Formulation 3 (Adju-Phos+Lysozyme); Group 4 was dosed with Formulation 4 (Lysozyme in PBS). Group 5 animals were naïve animals and did not receive any test article. On Day 14, each animal received a booster injection of the appropriate test article. On Day 28, animals were exsanguinated via cardiac puncture. The blood was collected into tubes containing no anticoagulant. The tubes were centrifuged at ˜2800 rpm for at least 10 minutes. Sera were placed into appropriately labeled tubes and stored at −16 to −22° C. until analysis by ELISA for chicken egg lysozyme IgG antibodies.

### Test Article Preparation

Group 1: SP-255a: Formulation 1 (Lysozyme+Liposomes): One vial was used per scheduled dosing time point for all animals in the dose group. Prior to injection, the vial was gently inverted at least 10 times and then gently swirled to obtain a homogenous mixture.
Group 2: SP-255b: Formulation 2 (Lyophilized Lysozyme+Liposomes): One lyophilized vial and one diluent vial was used per scheduled dosing time point for all animals in the dose group. Prior to injection, the lyophilized cake was reconstituted with 1 mL of diluent. The reconstituted vial was swirled to obtain a homogenous mixture.
Group 3: SP-256a, Formulation 3 (Lysozyme+Adju-Phos): One vial was used per scheduled dosing time point for all animals in the dose group. The vial was vigorously shaken to obtain good homogeneity.
Group 4: SP-256b, Formulation 4 (Lysozyme in 10% Sucrose): One vial was used per scheduled dosing time point for all animals in the dose group. Prior to injection, the vial was gently inverted at least 10 times and then gently swirled to obtain a homogenous mixture.
Group 5: Naïve animals; no test article was administered.

### Dosing Procedure

Four groups of 4 CD-1 female mice were dosed intramuscularly with 100 μL of test article on Day 0 and Day 14. Serum was collected from each animal on Day 28. Additionally, serum was collected from 4 naïve female CD-1 mice on Day 28 as a control group. The serum was frozen and stored at −80° C. until analysis by ELISA for chicken egg lysozyme IgG antibodies.

### Results and Discussion

### Characterization of Antibody Response to Each Formulation by Indirect ELISA

FIG. 1 shows an exemplary immune response (450 nm absorbance) of the various formulations as compared at different dilutions of the sera—80,000, 160,000, and 320.000-fold. The amount of absorbance at 450 nm reflects the amount of antibody present in the sera. FIG. 2 shows an exemplary Standard Curve of Mouse Anti-Lysozyme Antibody, while FIG. 3. shows an exemplary Average Amount of Mouse Anti-Lysozyme Antibody for a Formulation (mg/mL).

The immune response of the various formulations was compared (FIG. 1) at different dilutions of the sera (80K, 160K, and 320K—the 640K and 1280K dilutions were too dilute and their results not used). The amount of absorbance at 450 nm reflects the amount of antibody present in the sera.

The amount of mouse anti-Lysozyme antibody from the immune response of each of the four formulations was determined using a standard curve for purified mouse anti-Lysozyme antibody (Raybiotech). A representative standard curve is shown in FIG. 2. The standard curves were similar for all of the four ELISA plates.

The average quantified immune response to the Lysozyme vaccines from each group of mice (n=4) and the corresponding standard deviations are shown in FIG. 2. The group of naive mice did not give any immune response as expected (negative control). As shown in FIG. 3, Lysozyme without adjuvant had the lowest amount of antibodies induced.

The liquid liposomal vaccine induced approximately three times the amount of antibodies as the Lysozyme without adjuvant. The lyophilized liposomes showed a six-fold increase in antibodies. The Adju-Phos vaccine had the highest immune response, a nine-fold increase from the Lysozyme alone. Characteristics of the various vaccines are summarized in Table 4.

TABLE 4
Mean particle diameters and antibody titers of the vaccines

| | Particle | |
|---|---|---|
| | diameter (nm) | Antibody titer |
| Vaccine | Mean ± SD | (mg/mL) |
| Adju-Phos/Lysozyme (liquid) | >2000 | 478 ± 136 |
| Liposomes/Lysozyme (liquid) | 707 ± 5 | 177 ± 68 |
| Liposomes/Lysozyme (lyophilized) | 667 ± 113 | 306 ± 207 |
| Lysozyme (liquid) | 8.4 ± 1.4 | 50 ± 50 |

A two-tail t-test performed to evaluate the significance between the lyophilized liposomal formulation and Adju-Phos formulation, as well as the liquid and lyophilized liposomal formulations, gave p-values of 0.06 and 0.11, respectively. This showed that there was no significant difference between lyophilized liposomal formulation and Adju-Phos formulation, as well as the liquid and lyophilized liposomal formulations at the 95% confidence limit.

The formulations, in order of increasing immunogenic response, are as follows:
Lysozyme formulation<liquid liposomal formulation<lyophilized liposomal formulation≈Adju-Phos formulation.

The results show that all the vaccine formulations were better than the control lysozyme solution, inducing a significant immune response in mice. The findings of this study clearly demonstrate that the freeze-stable liposomal vaccine can be as immunogenic as an Aluminum-based vaccine. In addition, the freeze stable liposomal vaccine can be lyophilized into a stable, dry product that has the potential to become a room temperature stable vaccine.

### Example 2

A positively charged liposomal adjuvant systems was made in a similar fashion as described in Example 1, except with the lipid blend molar ratio of 40:25:20:15 containing DPPC:DOPC:cholesterol:SA. Recombinant tetanus light chain (TLC) was entrapped and/or associated with the lipid blend by hydration in the protein solution. The solution was subjected to three freeze-thaw cycles and extruded through 800 nm pore nucleopore membranes. The free unassociated TLC was removed by dialysis and the liposomal TLC complex was diluted to around 200 ng/ml protein. As a control, a non-adjuvant solution of just the TLC solution was injected into mice. All the solutions were dosed at 10 ng TLC per mouse. One intramuscular injection of 50 ul/mouse was administered into 5 mice per group and serum was collected following two weeks after the second booster injection of the test articles. Immunogenicity to the Tetanus Toxoid was evaluated using the mouse Anti-Tetanus Toxoid IgG ELISA Kit that detects and quantifies tetanus toxoid-specific IgG in mouse serum of vaccinated or immunized animals (ELISA Kit Cat No. 930-130-TMG, Alpha Diagnostic Internation, San Antonio, Tex., USA).

Results from the mouse immunogenicity study are shown in the FIGS. 4 and 5, where the average titers of mouse anti-Tetanus Toxid IgG from 5 mice are compared among the formulations (FIG. 5).

The results clearly show that the cationic liposomes containing TLC in both the liquid formulation that had been freeze-thawed three times and the lyophilized formulation that had been frozen to −40° C. and lyophilized gave a significant immune response and generated IgG antibodies to Tetanus Toxoid as measured by ELISA. The naïve and TLC solution without adjuvants did not give a significant immune response against the Tetanus Toxoid.

TABLE
Mean particle size of cationic liposomes-TLC

| | Particle Diameter (nm) |
|---|---|
| Formulation | Mean ± SD |
| Liquid cationic liposomes-TLC | 483 ± 15 |
| Lyophilized cationic liposomes- | 457 ± 108 |
| TLC | |

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A composition, comprising:
Dipalmitoyl phosphatidlcholine (DPPC),
Dioleoyl phosphatidylcholine (DOPC),
Cholesterol (Chol); and
a charged lipid with an entrapped or adsorbed protein antigen induce antibody response against an antigen.

2. The composition of claim 1, comprising liposomes or nano particles used as a vaccine adjuvant.

3. The composition of claim 1, comprising liposomes or nano particles which do not lose their immunogenicity after being exposed to freezing temperature during multiple freeze-thaws (freeze-stable adjuvants).

4. The composition of claim 1, wherein the DPPC, DOPC and Chol have a molar ratio of 40:25:20 of DPPC:DOPC:Chol and 15 for a negatively charged lipid Dipalmitoyl phosphatidylglycerol (DPPG).

5. The composition of claim 1, comprising negatively charged lipids including DOPA or DOPS.

6. The composition of claim 1, wherein the DPPC, DOPC and Chol have a molar ratio of 40:25:20 of DPPC:DOPC:Chol and 15 for a positively charged lipid Stearylamine (SA).

7. The composition of claim 1, wherein a mean hydrodynamic particle diameter of particles is in the size range of 300-1000 nm.

8. The composition of claim 1, wherein a solution containing the particles 1 is immunogenic after lyophilization when frozen to −40° C. to −50° C.

9. The composition of claim 1, wherein resulting adjuvant particles are used as an alternative to freeze-sensitive aluminum salt adjuvants in vaccine formulations.

10. The composition of claim 1, comprising a protein antigen selected from or derived from a group consisting of rotavirus, foot and mouth disease virus, influenza A virus, influenza B virus, influenza C virus, H1N1, H2N2, H3N2, H5N1, H7N7, H1N2, H9N2, H7N2, H7N3, H10N7, human parainfluenza type 2, herpes simplex virus, Epstein-Barr virus, varicella virus, porcine herpesvirus 1, cytomegalovirus, lyssavirus, Bacillus anthracis, anthrax PA and derivatives, poliovirus, Hepatitis A, Hepatitis B, Hepatitis C, Hepatitis E, distemper virus, Venezuelan equine encephalomyelitis, feline leukemia virus, reovirus, respiratory syncytial virus, Lassa fever virus, polyoma tumor virus, canine parvovirus, papilloma virus, tick borne encephalitis virus, rinderpest virus, human rhinovirus species, Enterovirus species, Mengovirus, paramyxovirus, avian infectious bronchitis virus, human T-cell leukemia-lymphoma virus 1, human immunodeficiency virus-1, human immunodeficiency virus-2, lymphocytic choriomeningitis virus, parvovirus B19, adenovirus, rubella virus, yellow fever virus, dengue virus, bovine respiratory syncitial virus, corona virus, Bordetella pertussis, Bordetella bronchiseptica, Bordetella parapertussis, Brucella abortis, Brucella melitensis, Brucella suis, Brucella ovis, Brucella species, Escherichia coli, Salmonella species, Salmonella typhi, Streptococci, Vibrio cholera, Vibrio parahaemolyticus, Shigella, Pseudomonas, tuberculosis, avium, Bacille Calmette Guerin, Mycobacterium leprae, Pneumococci, Staphylococci, Enterobacter species, Rochalimaia henselae, Pasteurella haemolytica, Pasteurella multocida, Chlamydia trachomatis, Chlamydia psittaci, Lymphogranuloma venereum, Treponema pallidum, Haemophilus species, Mycoplasma bovigenitalium, Mycoplasma pulmonis, Mycoplasma species, Borrelia burgdorferi, Legionalla pneumophila, Colstridium botulinum, Corynebacterium diphtheriae, Yersinia entercolitica, Rickettsia rickettsii, Rickettsia typhi, Rickettsia prowsaekii, Ehrlichia chaffeensis, Anaplasma phagocytophilum, Plasmodium falciparum, Plasmodium vivax, Plasmodium malariae, Schistosomes, trypanosomes, Leishmania species, Filarial nematodes, trichomoniasis, sarcosporidiasis, Taenia saginata, Taenia solium, Leishmania, Toxoplasma gondii, Trichinella spiralis, coccidiosis, Eimeria tenella, Cryptococcus neoformans, Candida albican, Apergillus fumigatus, coccidioidomycosis, Neisseria gonorrhoeae, malaria circumsporozoite protein, malaria merozoite protein, trypanosome surface antigen protein, pertussis, alphaviruses, adenovirus, diphtheria toxoid, tetanus toxoid, meningococcal outer membrane protein, streptococcal M protein, Influenza hemagglutinin, cancer antigen, tumor antigens, toxins, Clostridium perfringens epsilon toxin, ricin toxin, pseudomonas exotoxin, exotoxins, neurotoxins, cytokines, cytokine receptors, monokines, monokine receptors, plant pollens, animal dander, and dust mites.

11. The composition of claim 1, comprising a vaccine against diphtheria, tetanus, pertussis (whooping cough), influenza, hepatitis B, botulinium toxin, anthrax, or combination vaccines such as, PedvaxHlB (Haemophilus b Conjugate and Meninococcal Protein Conjugate), COMVAX® (Meningococcal Protein Conjugate and Hepatitis B, recombinant antigens), TRIPEDIA® (Diphtheria and Tetanus Toxoids and Acellular Pertussis antigens). INFANRIX (Diphtheria and Tetanus Toxoids and Acellular Pertussis antigens), or a vaccine that loses potency upon freezing.
